(19) United States
(12) Patent Application Publication
(10) Pub. No.: US 2025/0073176 A1
(43) Pub. Date: Mar. 6, 2025

(54) POLY(AMINO ACID) BASED CAPSULES

(71) Applicant: AGFA-GEVAERT NV, Mortsel (BE)

(72) Inventors: Johan Loccufier, Mortsel (BE); Jos Louwet, Mortsel (BE)

(73) Assignee: AGFA-GEVAERT NV, Mortsel (BE)

(21) Appl. No.: 18/368,373

(22) PCT Filed: Mar. 14, 2022
(86) PCT No.: PCT/EP2022/056500

§ 371 Date: Sep. 14, 2023

(30) Foreign Application Priority Data
Mar. 18, 2021 (EP) 21163396.1

(51) Int. Cl. A61K 9/50

(52) U.S. Cl. CPC A61K 9/5031

(57) ABSTRACT

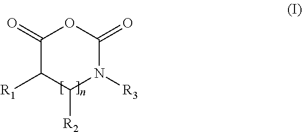

A capsule consisting of a polymeric shell surrounding a core, the core comprises an organic compound, the polymeric shell comprises a poly(amino acid) and is obtainable by interfacial polymerization of a N-carboxy-anhydride monomer according to general structure (I). The organic compound is a marine oil, a vegetable oil, an essential oil, a fragrance, a flavour, an insect repellent, a flame retardant, an active pharmaceutical ingredient or an agrochemical.

### TECHNICAL FIELD

It is an object of the invention to provide a poly(amino acid) based capsule.

It is a further object of the invention to provide a synthetic method for the preparation of poly(amino acid) based capsules.

### BACKGROUND ART

Biodegradability of polymers is an ever increasing demand in a whole set of applications, especially those applications holding the risk of polymers ending up in the environment. Therefore, more and more bio-based approaches are appearing in different fields of technology. Encapsulation is a very promising technology for controlled release of different chemicals, e.g. biological active products or fragrances, for protection of hydrolytically sensitive compounds in aqueous formulations and for separating reactivity in single fluid formulations. Amongst others, life sciences, agrochemicals and cosmetics are major fields of application for encapsulation, where release of encapsulated chemistry in the environment or contact with a biological environment is unavoidable. Therefore, biodegradability and biocompatibility will become an absolute requirement in all of these applications.

Nano-and microcapsules can be prepared using both chemical and physical methods. Encapsulation methodologies include complex coacervation, liposome formation, spray drying and precipitation and polymerisation methods. For technological applications, interfacial polymerisation is a particularly preferred technology, which has been reviewed by Zhang Y. and Rochefort D. (Journal of Microencapsulation, 29(7), 636-649 (2012) and by Salaün F. (in Encapsulation Nanotechnologies, Vikas Mittal (ed.), chapter 5, 137-173 (Scrivener Publishing LLC (2013)).

Polymerization methods are particularly preferred, as they allow the highest control in designing the capsules. More preferably interfacial polymerization and most preferably interfacial polycondensation is used to prepare the capsules for technological applications. In interfacial polymerization, polymerization occurs at the interface of the oil drops in an oil-in-water emulsion or at the interface of the water drops in water-in-oil emulsions. In, interfacial polycondensation, two reactants meet at the interface of the emulsion droplets and react rapidly.

In general, interfacial polymerisation requires the dispersion of an oleophilic phase in an aqueous continuous phase or vice versa. Classically each of the phases contains at least one dissolved monomer (a first shell component) that is capable of reacting with another monomer (a second shell component) dissolved in the other phase. Upon polymerisation, a polymer is formed that is insoluble in both the aqueous and the oleophilic phase. As a result, the formed polymer has a tendency to precipitate at the interface of the oleophilic and aqueous phase, hereby forming a shell around the dispersed phase, which grows upon further polymerisation.

Interfacial polymerisation technologies known in the prior art rely on the polymerisation of often petrochemical based synthetic monomers, leading to shell chemistry typically selected from polyamides, polyurea, polyurethanes, polyesters, polycarbonates or combinations thereof. Polycondensation products of aldehydes and other monomers such as melamine or urea are also well documented in the literature. However, in general all of this shell chemistry leads to non or scarcely degradable polymers.

Poly(amino acids) are a well-known class of biocompatible and biodegradable polymers and would be a preferred class of shell polymers for biocompatible micro-and nanocapsule design. However, classical interfacial polycondensation as described above, is not suited as preparation method for preparing poly(amino acid) based capsules.

Poly(amino acids) can be prepared by the polymerization of N-carboxy-anhydride monomers (NCA's) in a heterogeneous water-solvent-system. Wang et al. (Journal of Biomedical Research Part B: Applied Biomaterials, 89B(1), 45-54 (2009)) described the preparation of glycopeptide microspheres starting from acylated chitosan as initiator for graft-polymerization of NCA's in a heterogeneous water-solvent mixture. The disclosed microspheres were prepared using L-leucine as amino acid. The spheres have a particle size of several tens of microns up to a few hundred microns and did not contain specific core material.

Jacobs et al. disclosed mini-emulsion polymerization using NCA's in a heterogeneous water-solvent-mixture (J. Am. Soc., 141, 12522-12526 (2019)). The particle size was in the range of 200 nm. However, the particles did not contain core material. Deformation of the particles due to secondary structuration of the particles was observed.

In a lot of approaches, amphiphilic block copolymers containing poly(amino acid) blocks are prepared separately and assembled into micelle like capsules or transferred into capsules using coacervation type of approaches. The self-assembly of amphiphilic block copolymers into micelles can hold up core material. Micelle based capsules have the disadvantage of a much weaker shell than a capsule with a polymeric shell. In many systems, a crosslinking of the shell of micellar systems is therefore required.

WO96/40279 discloses the production of microspheres via cavitation of amphiphilic poly amino acid block co-polymers. Stable microspheres can only be achieved for a certain hydrophobic-hydrophilic balance of the block co-polymers, hence limiting the number of suitable amino acid polymers considerably.

In the literature, for example, Jianxun Ding in Nanotechnology 22 (2011) 494012, different block copolymer based micelles have been documented as encapsulation technology. However, this approach requires a first step of separate synthesis of the amphiphilic block copolymers, which needs to be thoroughly controlled and adjusted towards the compound or functionality that needs to be encapsulated. In a second step the micelles are formed in a liquid medium. This process has to be repeated for every different functionality to be encapsulated. By their nature, micellar approaches are vulnerable towards different process conditions (pH, ionic strength, . . . of the aqueous medium), limiting the latitude for industrialization.

Micelle like capsules mostly need a liquid medium to retain its spherical structure such as to hold the core material in the inside of the micelle. Isolation of the micelle in a dried state is hence very difficult or not possible. Micelle like capsules have a limited range of obtainable particle size in contrast to capsules obtained by interfacial polymerization, more particularly in the lower particle size range. Furthermore, the approaches by means of amphiphilic block copolymers allow good control on the polymer structure but require exhaustive synthetic procedures to prepare the well-defined polymers, making them less suitable for technical applications in contrast to interfacial polymerization based technologies.

Other encapsulation technologies such as e.g. complex coacervation, require thorough control of the operational process window, which is often quite narrow, limiting the flexibility of the technology on an industrial scale.

Therefore, there is still a need for encapsulation approaches for the design of poly(amino acid) based capsules having a wide variety of particle sizes, having a mechanical strong shell, which can be isolated in a dry state and which can be obtained via a single step process.

### SUMMARY OF INVENTION

Now it has been found that poly(amino acid) based core shell structures, obtained by interfacial ring opening polymerization of monomers according to general structure I, can realize the objects of the present invention.

The present invention comprises capsules consisting of a polymeric shell based on poly(amino acids) surrounding a core as defined in claim 1.

According to another aspect, the present invention includes a method of preparing the capsules of claim 1. This method is defined in claim 11.

Other features, elements, steps, characteristics and advantages of the present invention will become more apparent from the following detailed description of preferred embodiments of the present invention. Specific embodiments of the invention are also defined in the dependent claims.

### DESCRIPTION OF EMBODIMENTS

### A. The Capsule

The objects of the present invention are realized by a core shell structure, wherein said core comprises an organic compound and said shell comprises an oligo-or poly(amino acid), obtained by oligomerization or polymerization of at least one N-carboxy-anhydride monomer according to general structure I

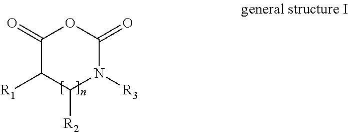

wherein

n represents 0 or 1

R₁, R₂ and R₃ are selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group

Any of R₁, R₂ and R₃ may represent the necessary atoms to form a five to eight membered ring.

Preferably, the organic compound is a substantially low volatile compound. Substantially low volatile is defined as having a boiling point of at least 150° C. at 1013 mPas.

More preferably, the organic compound is a hydrophobic compound, meaning, having an octanol-water partition coefficient, expressed as log K_{ow} of at least 0.3. Without being bound by any theory, it is thought that a hydrophobic compound in the oleophilic drops during the interfacial polymerization keeps the formed poly(amino acid) chains having a hydrophilic character, to the outside of the drops resulting in a strong and dense sphere polymeric shell.

The particle size of the capsules of the invention is preferably from 0.05 μm to 10 μm, more preferably from 0.07 μm to 5 μm and most preferably from 0.1 μm to 3 μm. Capsules according to the present invention having a particle size below 1 μm are particularly preferred.

A.1. The N-carboxy-anhydryde Monomer

In a preferred embodiment n represents 0. In a particular preferred embodiment R₃ represents a hydrogen or an alkyl group, a hydrogen being the most preferred.

In another preferred embodiment R₁ and R₂ are selected from the group consisting of a hydrogen, a substituted or unsubstituted alkyl group, a substituted or unsubstituted aralkyl group, a substituted or unsubstituted alkaryl group and a substituted or unsubstituted aryl group.

In further preferred embodiment, the N-carboxy-anhydride monomer according to general structure is selected from the group consisting of a glycine derivative, an alanine derivative, a leucine derivative, a phenylalanine derivative, a phenylglycine derivative, a valine derivative, a glutamic acid derivative, an aspartic acid derivative, a lysine derivative, an ornithine derivative, a histidine derivative, a methionine derivative, a cysteine derivative, an arginine derivative, a tryptophane derivative, a cysteine derivative, an isoleucine derivative, a tyrosine derivative, a proline derivative and a serine derivative. Both D-and L-amino acid derivatives and mixtures thereof can be used.

Typical N-carboxy-anhydride monomers are given in Table 1 without being limited thereto.

TABLE 1

| | |
|---|---|
| 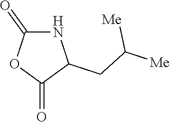 [SMILES: CC(C)CC1NC(=O)OC1=O] | NCA-1 |
| 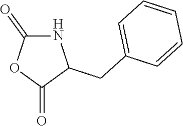 [SMILES: O=C1NC(Cc2ccccc2)C(=O)O1] | NCA-2 |
| 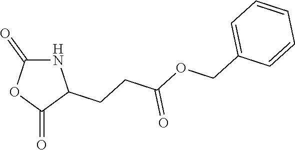 [SMILES: O=C(CCC1NC(=O)OC1=O)OCc1ccccc1] | NCA-3 |
| 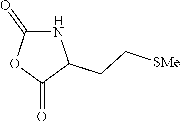 [SMILES: CSCCC1NC(=O)OC1=O] | NCA-4 |
| 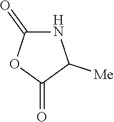 | NCA-5 |
| 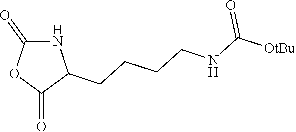 [SMILES: CC(C)(C)OC(=O)NCCCCC1NC(=O)OC1=O] | NCA-6 |
| 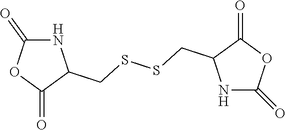 [SMILES: O=C1NC(CSSCC2NC(=O)OC2=O)C(=O)O1] | NCA-7 |
| 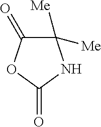 | NCA-8 |
| 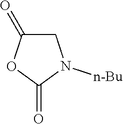 | NCA-9 |
| 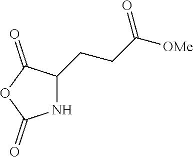 [SMILES: COC(=O)CCC1NC(=O)OC1=O] | NCA-10 |
| 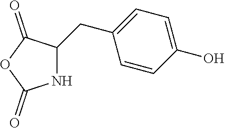 [SMILES: O=C1NC(Cc2ccc(O)cc2)C(=O)O1] | NCA-11 |
| 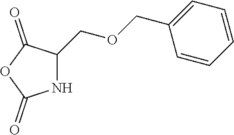 [SMILES: O=C1NC(COCc2ccccc2)C(=O)O1] | NCA-12 |

N-carboxy-anhydrides (NCA's) have been prepared using different synthetic methodologies, starting with the oldest method, known as Leuchs' method, starting from chloroformate acylation of the amino acid, followed by conversion to the corresponding NCA via its acid chloride. Several variants have been published on this methods, by Wessely and by Katchalski, respectively using a mixed anhydride method and a conversion using PBr₃. Probably, the most well-known method is the Fuchs-Farting method, using phosgene for direct conversion of the amino acid to the corresponding NCA. For safety reasons, phosgene has been replaced by di- or triphosgene in later research. Over the last years, several phosgene free methodologies have been disclosed. The methodologies have been reviewed by Secker et al. (Macromol. Biosci., 15, 881-891 (2015)).

### A.2. The Encapsulation Method

The capsules according to the present invention are prepared using a ring opening polymerization method, more preferably using interfacial ring opening polymerization.

The interfacial polymerization method according to the invention allows the preparation of capsules in a single step process and over a broad scope of functionalities and particle sizes, making it especially suitable for an industrial production process, more particularly for a continue industrial process. By simply adjusting the monomer ratios, the technology can easily be tuned towards the functionality to be encapsulated and the physical properties can easily be adjusted towards different applications without major changes in the process conditions leading to a robust technology with considerable latitude towards industrialization.

The ring opening polymerization of N-carboxy-anhydrides has been reviewed by Cheng and Deming (Top. Curr. Chem., 310, 1-26 (2012)). Primary and optionally secondary amines are the most obvious initiators and are widely used to initiate the ring opening polymerization via nucleophilic initiation. Basic initiators can initiate the ring opening polymerization via an activated monomer mechanism, starting by deprotonation of the NCA's followed by ring opening polymerization. When amine initiators are used, both mechanisms often run in parallel. Transition metal initiation is known to give better control on the polymerization. The use of hexamethyldisilazane as initiator has also been disclosed for better controlling the polymerization.

In a further preferred embodiment, a mixture of N-carboxy-anhydrides, derived from different amino acids is used. In an even further embodiment, a mixture of different chirality is used, preferably in a 9/1 to 1/9 ratio of a mixture of D- and L-amino acids. In another preferred embodiment, a mixture of chirality and different amino acids are used. Mixing D- and L-amino acids prevents the poly(amino acid) to form a secondary or tertiary structure as peptides do in nature. The obtained polymeric shell is hence denser and mechanically more resistant.

In a particularly preferred interfacial ring opening polymerization method for the preparation of the capsules according to the present invention, the N-carboxy-anhydride monomers and core material are dissolved in a substantially water immiscible solvent and emulsified in an aqueous solution containing a polymerization initiator. Upon emulsifying and optionally removing said substantially water immiscible solvent, the ring opening polymerization is initiated at the interface. Upon propagation, a poly(amino acid) shell is formed at the organic-water interface, generating a core-shell structure, encapsulating a functional component or a functional formulation. The obtained polymeric shell is mechanically strong and stable and allows the capsule to be isolated from the liquid wherein the capsules have been prepared.

The functional component or functional formulation is preferably an organic compound. The organic compound is a hydrophobic compound, meaning, having an octanol-water partition coefficient, expressed as log K_{ow} of at least 0.3.

If the core material is a liquid, dissolving in a substantially water immiscible solvent can be omitted and the NCA's can be directly dissolved in the core material. The capsules according to the present invention are particularly suited to hold up liquid core material. Micellar based capsules are much less suited to encapsulate and hold up liquid core material. Indeed, the shell of a micellar system is in many cases too permeable with respect to a polymeric shell obtained by the encapsulation method of the invention.

A particularly preferred interfacial ring opening polymerization method comprises the steps of
a) dissolving a compound according to general structure I and an organic compound in a water immiscible solvent; and
b) dissolving a polymerization initiator in an aqueous liquid; and
c) emulsifying the solution obtained in step a) into the aqueous liquid; and
d) optionally evaporating the water immiscible solvent; and
e) polymerizing the compound according to general structure I

The particle size of the capsules of the invention is modified by modifying the emulsification technology, the use of an emulsification aid and the ratio of an emulsification aid to the shell and core during emulsification, the nature of the emulsification aid, changing the viscosity of the continuous or dispersed phase, the ratio of the continuous and dispersed phase, the nature of the core content and the nature of the shell monomers. High shear technologies and ultrasound based technologies are particularly preferred as emulsification technologies. The particle size of the capsules according to the present invention can be tuned by tuning the shear in high shear technologies or by changing the power and amplitude upon sonification.

Preferably, the organic compound has an octanol-water partition coefficient, expressed as log K_{ow} of at least 0.3

Di- or multifunctional primary or secondary amines or mixtures thereof are particularly preferred initiators for the ring opening polymerization of the NCA's. The initiators are water soluble and can be functionalized with additional hydrophilic functional groups, preferably selected from the group consisting of a carboxylic acid or salt thereof, a sulfonic acid or salt thereof, a phosphonic acid or salt thereof, a phosphate ester or salt thereof, a sulfate ester or salt thereof, a poly-hydroxyl functionalized group, a poly(ethylene glycol), an ammonium group, a sulfonium group and a phosphonium group.

The incorporation of a poly(ethylene gycol) functional group is particularly useful to give stealth properties to the capsules of the invention if used as drug delivery system in the human or animal body. These stealth properties are required to avoid uptake by the reticuloendothelial system and only release drug at the required site in a controlled manner. Typical initiators are given in Table 2 without being limited thereto.

TABLE 2

| | |
|---|---|
| 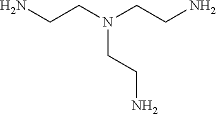 | INI-1 |
| 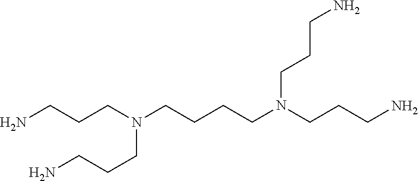 [SMILES: NCCCN(CCCN)CCCCN(CCCN)CCCN] | INI-2 |
| 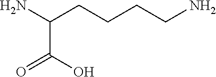 | INI-3 |
| 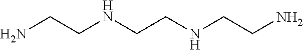 | INI-4 |
| 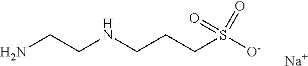 [SMILES: NCCNCCCS(=O)(=O)[O-] ; [Na+]] | INI-5 |
| 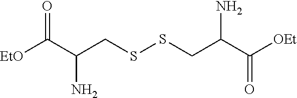 [SMILES: CCOC(=O)C(N)CSSCC(N)C(=O)OCC] | INI-6 |
| 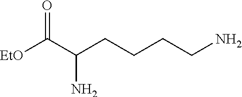 [SMILES: CCOC(=O)C(N)CCCCN] | INI-7 |
| 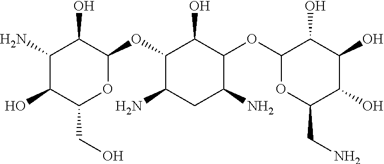 [SMILES: NC[C@H]1OC(OC2[C@@H](N)C[C@@H](N)[C@H](O[C@H]3O[C@H](CO)[C@@H](O)[C@H](N)[C@H]3O)[C@H]2O)[C@H](O)[C@@H](O)[C@@H]1O] | INI-8 |
| 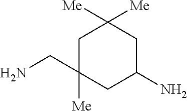 [SMILES: CC1(C)CC(N)CC(C)(CN)C1] | INI-9 |
| 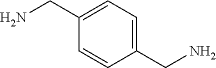 | INI-10 |
| 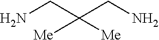 | INI-11 |
| 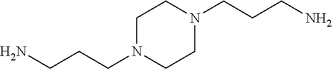 [SMILES: NCCCN1CCN(CCCN)CC1] | INI-12 |
| 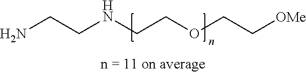 | INI-13 |
| 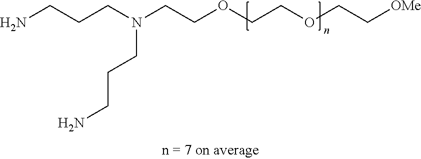 | INI-14 |

In a further preferred embodiment, the shell composition further comprises a crosslinker. After biocompatibility and biodegradability, one of the most basic requirements of a capsule is stability in the medium wherein it has to function or has to be stored, e.g. the human body for a drug delivery system. If a system is not stable in its medium, this could result in a preliminary burst release of the payload or in non-targeted areas. Increased stability results in increased storage stability and for drug delivery systems, in an increased blood circulation time and increased bioavailability. With a crosslinker, the stability and mechanical resistance of the shell of the capsule can be modified to meet the specifications of the system in which the capsule is used. Further, the use of a crosslinker makes it possible to precisely control the drug release in the use of a drug delivery purpose of the capsules of the invention.

Any crosslinker known to crosslink amine functionalized polymers can be used. Preferred crosslinkers are selected from the group consisting of di- or multifunctional isocyanates, di- or multifunctional β-keto-esters, di- or multifunctional β-keto-amides, di- or multifunctional 1,3-diketones, di- or multifunctional epoxides or oxetanes, di- or multifunctional anhydrides, di- or multifunctional N-carboxy-anhydrides, di-or multifunctional Michael acceptors such as acrylates, methacrylates, maleimides, vinyl sulfones and the like and di-or multifunctional five membered carbonates.

Preferably, an additional emulsification aid is used during the emulsification step. Typical emulsification aids are selected from polymers and surfactants. The polymers and surfactants can be co-reactive polymers or surfactants, e.g. functionalized with primary and secondary amines, taking the role of both initiator and emulsification aid, leading to so called self-dispersing capsules. The surfactant can be anionic, non-ionic, cationic or zwitterionic. As stabilizing polymers, hydroxyl functionalized polymers are particularly preferred, preferably selected from polysaccharides and poly(vinyl alcohol) or poly(vinyl alcohol) copolymers or derivatives thereof.

### B. Fields of Application

The encapsulation technology, disclosed in the present invention is particularly useful in the field of personal care, pharmaceuticals, nutrition, agrochemicals and household applications, especially for controlling the release of the active components or protecting the active components from hydrolysis or oxidation. Examples are encapsulation of food ingredients, probiotics, fragrances and flavours, agrochemicals, flame retardants and last but not least, active pharmaceutical ingredients.

More general, the component in the core of the capsule preferably has an octanol-water partition coefficient, expressed as log K_{ow} of at least 0.3, more preferably of at least 0.5 and most preferably of at least 1.

The octanol-water partition coefficient is defined as follows,

$${K}_{ow} = {C}_{op} / {C}_{w}$$

where Cₒₚ and C_{w} are the concentrations of the compound under consideration in g L⁻¹ at 25° C., respectively in the octanol rich phase and the water rich phase.

The encapsulation technology according to the present invention is particularly of interest for the encapsulation of substantially non-reactive hydrophobic components such as marine oils, vegetable oils, and essential oils. The technology is also particularly of interest for the encapsulation of fragrances, flavors and insect repellents.

The encapsulation technology according to the present invention is further particularly of interest for the encapsulation of active pharmaceutical ingredients and agrochemicals.

More particularly, the encapsulation technology is useful in the encapsulation of active pharmaceutical ingredients such as an anti-cancer drug, a vaccine, a peptide, a protein, a sonosensitizer, a carrier for a drug, a gene, a growth factor such as recombinant bone morphogenetic protein (rhBMP-2), progesterone, procaine hydrochloride, bovine serum albumin, benzocaine, insulin, etc. The capsules of the invention are particularly suitable to be incorporated in a pharmaceutical composition for the treatment of cancer.

Capsules of the invention can be used in the treatment of cancer such as embolotherapy as disclosed in EP2891485A. These microspheres in an embolotherapy are used in a liquid when inserted into the human body, but are preferably maintained in a solid state for stable storage. In another aspect of the invention, the capsules of the invention are suitable in sonodynamic treatment of a metastatic disease, micrometastatic disease, or in the treatment of multiple primary tumours.

For use in any of the medical treatment methods described above, the capsules of the invention will generally be provided in a pharmaceutical composition together with at least one pharmaceutically acceptable carrier or excipient. Such pharmaceutical compositions may be formulated using techniques well known in the art. The route of administration will depend on the intended use. Typically, these will be administered systemically and may thus be provided in a form adapted for parenteral administration, e.g. by intradermal, subcutaneous, intraperitoneal or intravenous injection.

Suitable pharmaceutical compositions include suspensions and solutions which contain the capsules of the invention together with one or more inert carriers or excipients. Suitable carriers include saline, sterile water, phosphate buffered saline and mixtures thereof. The compositions may additionally include other agents such as emulsifiers, suspending agents, dispersing agents, solubilisers, stabilisers, buffering agents, wetting agents, preserving agents, etc. The pharmaceutical compositions may be sterilised by conventional sterilisation techniques. Solutions containing the particles may be stabilised, for example by the addition of agents such as viscosity modifiers, emulsifiers, solubilising agents, etc.

Preferably, the pharmaceutical compositions will be used in the form of an aqueous suspension or dispersion of the capsules in water or a saline solution, e.g, phosphate-buffered saline. The particles may be supplied in the form of a lyophilised powder for reconstitution at the point of use, e.g. for reconstitution in water, saline or phosphate-buffered saline.

The capsule according to the invention is particularly useful in a consumer product which is selected from the group consisting of a shampoo, a hair conditioner, a hair rinse, a hair refresher, a hair fixative or styling aid, a hair bleach, a hair dye or colorant, a soap, a body wash, a cosmetic preparation, an all-purpose cleaner, a bathroom cleaner, a floor cleaner, a window cleaner, a bath tissue, a paper towel, a disposable wipe, a diaper rash cream or balm, a baby powder, a diaper, a bib, a baby wipe, an oral care product, a tooth paste, an oral rinse, an tooth whitener, a denture adhesive, a chewing gum, a breath freshener, an orally dissolvable strips, a chewable candy, a hard candy, a hand sanitizer, an anti-inflammatory balm, an anti-inflammatory ointment, an anti-inflammatory spray, a health care device, a dental floss, a toothbrush, a tampon, a feminine napkin, a personal care product, a sunscreen lotion, a sunscreen spray, a wax-based deodorant, a glycol type deodorant, a soap type deodorant, a facial lotion, a body lotion, a hand lotion, a body powder, a shave cream, a bath soak, an exfoliating scrub, a foot cream, a facial tissue, a cleansing wipe, a fabric care product, a fabric softener, a fabric refresher, an ironing water, a liquid laundry detergent, a liquid dish detergent, an automatic dish detergent, a unit dose tablet or capsule, a scent booster, a drier sheet, a fine fragrance, a solid perfume, a powder foundation, a liquid foundation, an eye shadow, a lipstick or lip balm, an Eau De Toilette product, a deodorant, a rug deodorizer, a candle, a room deodorizer, a disinfectant, an anti-perspirant, an roll-on product, and an aerosol product.

### C. EXAMPLES

### C.1. Materials

All compounds are supplied by TCI Europe unless otherwise specified.
L-phenylalanine N-carboxy anhydride, D-phenylalanine N-carboxy anhydride and D,L-phenylalanine N-carboxy anhydride can be prepared according to standard methods as disclosed by Gabashvill et al. (Journal of Physical Chemistry B, 111(38), 11105-11110 (2007)) and Otake et al. (Angewandte Chemie, International Edition, 57 (35), 11389-11393 (2018)).
L-leucine N-carboxy anhydride, D-leucine N-carboxy anhydride and D,L-leucine N-carboxy anhydride can be prepared according to standard methods as disclosed by Baars et al. (Organic Process Research and Development, 7(4), 509-513 (2003)).
L-methionine N-carboxy anhydride and D,L-methionine N-carboxy anhydride can be prepared according to standard methods as disclosed by Verdie at al. (Chemistry-An Asian Journal, 6(9), 2382-2389 (2011).
γ-benzyl-L-glutamate N-carboxy anhydride can be prepared according to standard methods as disclosed by Wang et al. (RSC Advances, 6(8), 6368-6377 (2016)).
Glyceryl tricaprate was supplied by Esterchem.
δ-undecalactone was supplied by SAF Bulk Chemicals.
Disflamol TKP is a mixture of cresyl and phenyl esters of phosphoric acid supplied by Albright & Wilson.
Mowiol 4 88 is a poly(vinyl alcohol) supplied by Kuraray.
Marlon A365 is an anionic surfactant supplied by Sasol Germany GMBH.
Tris(2-aminoethyl)amine was supplied by TCl.
Tracer-1 is a fluorescent marker according to the following structure (CASRN917102-92-2) and can be prepared according to methods disclosed in WO2008056506 (Konica Minolta Medical & Graphic Inc.).

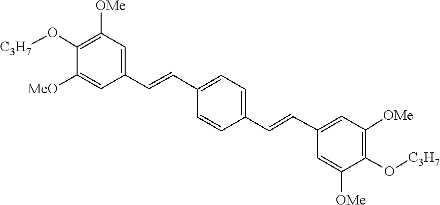

Crosslinker-1 is a trifunctional β-keto-ester according to the following structure, which can be prepared as disclosed by Speisschaert et al. (Polymer, 172, 239-246 (2019)).

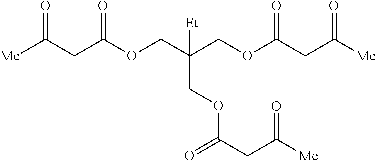 [SMILES: CCC(COC(=O)CC(C)=O)(COC(=O)CC(C)=O)COC(=O)CC(C)=O]

Takenate D120N is a trifunctional isocyanate supplied by Mitsui.
Desmodur N75BA is a trifunctional isocyanate supplied by Covestro.
CATSURF-1 is a cationic surfactant according to the following structure, which can be prepared as disclosed in WO2018137993 (Agfa N.V) as Surf-3.

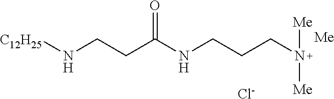

### C.2. Methods

The particle size of the capsules was measured using a Zetasizer™ Nano-S (Malvern Instruments, Goffin Meyvis).

### C.3. Example 1

This example illustrates the encapsulation of different chemicals using the interfacial ring opening polymerization according to the present invention.

### The Synthesis of INVCAP-1 to INVCAP-3

### Encapsulation of Glyceryl Tricaprate (INVCAP-1)

A first solution was prepared by dissolving 2.5 g L-phenylalanine N-carboxy anhydride, 0.25 g D-phenylalanine N-carboxy anhydride, 0.25 g D,L-phenylalanine N-carboxy anhydride, 0,303 g crosslinker-1, 2,8 g glyceryl tricaprate and 100 mg Tracer-1 in 18 ml ethyl acetate.

A second solution was prepared by dissolving 0.684 g Mowiol 4 88, 0.256 g Marlon A365 and 0.115 g tris(2-aminoethyl) amine in 30 ml water.

The first solution was added to the second solution using mixing with an Ultra Turrax T25 (IKA) at 6000 rpm for 5 minutes while maintaining the temperature of the emulsion between 20 and 30° C. 10 ml water was added followed by evaporation of the mixture under reduced pressure to 20 g. The polymerization was allowed to continue at room temperature for 24 hours.

The measured average particle size was 1.01 μm.

### The Encapsulation of δ-Undecalactone (INVCAP-2)

A first solution was prepared by dissolving 2.5 g L-phenylalanine N-carboxy anhydride, 0.25 g D-phenylalanine N-carboxy anhydride, 0.25 g D,L-phenylalanine N-carboxy anhydride, 0,303 g crosslinker-1, 2,8 g δ-undecalactone and 100 mg Tracer-1 in 18 ml ethyl acetate.

A second solution was prepared by dissolving 0.684 g Mowiol 4 88, 0.256 g Marlon A365 and 0.115 g tris(2-aminoethyl) amine in 30 ml water.

The first solution was added to the second solution using mixing with an Ultra Turrax T25 (IKA) at 6000 rpm for 5 minutes while maintaining the temperature of the emulsion between 20 and 30° C. 10 ml water was added followed by evaporation of the mixture under reduced pressure to 20 g. The polymerization was allowed to continue at room temperature for 24 hours.

The measured average particle size was 1.95 μm.

### The Encapsulation of Disflamoll TKP (INVCAP-3)

A first solution was prepared by dissolving 2.5 g L-phenylalanine N-carboxy anhydride, 0.25 g D-phenylalanine N-carboxy anhydride, 0.25 g D,L-phenylalanine N-carboxy anhydride, 0,303 g crosslinker-1, 2,8 g Disflamoll TKP and 100 mg Tracer-1 in 18 ml ethyl acetate.

A second solution was prepared by dissolving 0.684 g Mowiol 4 88, 0.256 g Marlon A365 and 0.115 g tris(2-aminoethyl) amine in 30 ml water.

The first solution was added to the second solution using mixing with an Ultra Turrax T25 (IKA) at 6000 rpm for 5 minutes while maintaining the temperature of the emulsion between 20 and 30° C. 10 ml water was added followed by evaporation of the mixture under reduced pressure to 20 g. The polymerization was allowed to continue at room temperature for 24 hours.

The measured average particle size was 1.25 μm.

### Characterization of INVCAP-1 to INVCAP-3

### Fluorescence Imaging

The inventive capsules INVCAP-1 to INVCAP-3 were analyzed using a light microscope at a magnification of 63× and equipped with a UV lamp, emitting UV in the wavelength 365 nm. First a visual image was taken of each sample. In a second image, the capsules dispersion was exposed to UV light and a fluorescence image was taken. An overlay was made between the visual and the fluorescence image. From the overlay, it became clear that the fluorescence image for all capsules perfectly match with the visual image of the particles, clearly indicating that the chemistry was encapsulated.

### Centrifugation

The inventive capsules INVCAP-1 to INVCAP-3 were isolated using centrifugation with a Thermo Scientific SL8 centrifuge at a rotation speed of 4500 RPM for one hour. Both the capsules and the supernatant were isolated and analyzed for the presence of non-encapsulated compounds and fluorescence. In none of the samples, non-encapsulated compounds could be detected. Fluorescence could only be detected in the capsules themselves, again clearly indicating that the chemistry was encapsulated.

The isolated capsules were re-dispersed in water and both a visual and fluorescence image was taken. Again the fluorescence and visual image for INVCAP-1 to INVCAP-3 perfectly matched.

INVCAP-1 to INVCAP-3 were dried to obtain a powder. No indication could be found for encapsulated chemistry found outside the capsules. The powders could easily be re-dispersed in water.

### C.4. Example 2

This example illustrates that a scope of amino acids can be used in the interfacial ring opening polymerization, to prepare capsules according to the present invention.

### Leucine as (Co)Monomer in the Capsule Shell (INVCAP-4 to INVCAP-7)

### The Synthesis of INVCAP-4

3 g L-leucine N-carboxy anhydride was dissolved in 18 ml ethyl acetate. The solution was filtered over a 1.7 micron filter. 0.303 g Crosslinker-1, 0.1 g Tracer-1 and 2.28 g glyceryl tricaprate were added.

A second solution was prepared by dissolving 0.684 g Mowiol 4 88, 0.256 g Marlon A365 and 0.115 g tris(2-aminoethyl) amine in 30 ml water.

The first solution was added to the second solution using mixing with an Ultra Turrax T25 (IKA) at 6000 rpm for 5 minutes while maintaining the temperature of the emulsion between 20 and 30° C. 10 ml water was added followed by evaporation of the mixture under reduced pressure to 20 g. The polymerization was allowed to continue at room temperature for 24 hours.

The measured average particle size was 0.86 μm.

### The Synthesis of INVCAP-5

1.5 g L-leucine N-carboxy anhydride and 1.5 g D-phenylalanine N-carboxy anhydride were dissolved in 18 ml ethyl acetate. The solution was filtered over a 1.7 micron filter. 0.336 g Crosslinker-1, 0.1 g Tracer-1 and 2.59 g glyceryl tricaprate were added.

A second solution was prepared by dissolving 0.692 g Mowiol 4 88, 0.259 g Marlon A365 and 0.127 g tris(2-aminoethyl) amine in 30 ml water.

The first solution was added to the second solution using mixing with an Ultra Turrax T25 (IKA) at 6000 rpm for 5 minutes while maintaining the temperature of the emulsion between 20 and 30° C. 10 ml water was added followed by evaporation of the mixture under reduced pressure to 25 g. The polymerization was allowed to continue at room temperature for 24 hours.

The measured average particle size was 0.574 μm.

### The Synthesis of INVCAP-6

0.75 g L-leucine N-carboxy anhydride, 0.75 g D-leucine N-carboxy anhydride, 0.75 g L-phenylalanine N-carboxy anhydride and 0.75 g D-phenylalanine N-carboxy anhydride were dissolved in 18 ml ethyl acetate. The solution was filtered over a 1.7 micron filter. 0.336 g Crosslinker-1, 0.1 g Tracer-1 and 2.59 g glyceryl tricaprate were added.

A second solution was prepared by dissolving 0.692 g Mowiol 4 88, 0.259 g Marlon A365 and 0.127 g tris(2-aminoethyl) amine in 30 ml water.

The first solution was added to the second solution using mixing with an Ultra Turrax T25 (IKA) at 6000 rpm for 5 minutes while maintaining the temperature of the emulsion between 20 and 30° C. 10 ml water was added followed by evaporation of the mixture under reduced pressure to 25 g. The polymerization was allowed to continue at room temperature for 24 hours.

The measured average particle size was 0.569 μm.

### The Synthesis of INCAP-7

1.5 g L-leucine N-carboxy anhydride and 1.5 g L-phenylalanine N-carboxy anhydride were dissolved in 18 ml ethyl acetate. The solution was filtered over a 1.7 micron filter. 0.336 g Crosslinker-1, 0.1 g Tracer-1 and 2.59 g glyceryl tricaprate were added.

A second solution was prepared by dissolving 0.692 g Mowiol 4 88, 0.259 g Marlon A365 and 0.127 g tris(2-aminoethyl) amine in 30 ml water.

The first solution was added to the second solution using mixing with an Ultra Turrax T25 (IKA) at 6000 rpm for 5 minutes while maintaining the temperature of the emulsion between 20 and 30° C. 10 ml water was added followed by evaporation of the mixture under reduced pressure to 30 g. The polymerization was allowed to continue at room temperature for 24 hours.

The measured average particle size was 0.465 μm.

### Methionine as (Co)Monomer in the Capsule Shell (INVCAP-8 to INVCAP-11)

### The Synthesis of INVCAP-8

3 g D,L-methionine N-carboxy anhydride was dissolved in 18 ml ethyl acetate. The solution was filtered over a 1.7 micron filter. 0.331 g Crosslinker-1, 0.1 g Tracer-1 and 2.33 g glyceryl tricaprate were added.

A second solution was prepared by dissolving 0.69 g Mowiol 4 88, 0.259 g Marlon A365 and 0.125 g tris(2-aminoethyl) amine in 30 ml water.

The first solution was added to the second solution using mixing with an Ultra Turrax T25 (IKA) at 6000 rpm for 5 minutes while maintaining the temperature of the emulsion between 20 and 30° C. 10 ml water was added followed by evaporation of the mixture under reduced pressure to 25 g. The polymerization was allowed to continue at room temperature for 24 hours.

### The Synthesis of INVCAP-9

1.5 g D,L-methionine N-carboxy anhydride, 0.75 g L-leucine N-carboxy anhydride and 0.75 g D-leucine N-carboxy anhydride were dissolved in 18 ml ethyl acetate. The solution was filtered over a 1.7 micron filter. 0.35 g Crosslinker-1, 0.1 g Tracer-1 and 2.32 g glyceryl tricaprate were added.

A second solution was prepared by dissolving 0.696 g Mowiol 4 88, 0.259 g Marlon A365 and 0.132 g tris(2-aminoethyl) amine in 30 ml water.

The first solution was added to the second solution using mixing with an Ultra Turrax T25 (IKA) at 6000 rpm for 5 minutes while maintaining the temperature of the emulsion between 20 and 30° C. 10 ml water was added followed by evaporation of the mixture under reduced pressure to 25 g. The polymerization was allowed to continue at room temperature for 24 hours.

### The synthesis of INVCAP-10

0.6 g D,L-methionine N-carboxy anhydride, 0.6 g L-leucine N-carboxy anhydride, 0.6 g D-leucine N-carboxy anhydride, 0.6 g L-phenylalanine N-carboxy anhydride and 0.6 g D-phenylalanine N-carboxy anhydride were dissolved in 18 ml ethyl acetate. The solution was filtered over a 1.7 micron filter. 0.335 g Crosslinker-1, 0.1 g Tracer-1 and 2.32 g glyceryl tricaprate were added.

A second solution was prepared by dissolving 0.696 g Mowiol 4 88, 0.259 g Marlon A365 and 0.132 g tris(2-aminoethyl) amine in 30 ml water.

The first solution was added to the second solution using mixing with an Ultra Turrax T25 (IKA) at 6000 rpm for 5 minutes while maintaining the temperature of the emulsion between 20 and 30° C. 10 ml water was added followed by evaporation of the mixture under reduced pressure to 25 g. The polymerization was allowed to continue at room temperature for 24 hours.

### The synthesis of INVCAP-11

0.6 g L-methionine N-carboxy anhydride, 0.6 g L-leucine N-carboxy anhydride, 0.6 g D-leucine N-carboxy anhydride, 0.6 g L-phenylalanine N-carboxy anhydride and 0.6 g D-phenylalanine N-carboxy anhydride were dissolved in 18 ml ethyl acetate. The solution was filtered over a 1.7 micron filter. 0.335 g Crosslinker-1, 0.1 g Tracer-1 and 2.32 g glyceryl tricaprate were added.

A second solution was prepared by dissolving 0.696 g Mowiol 4 88, 0.259 g Marlon A365 and 0.132 g tris(2-aminoethyl) amine in 30 ml water.

The first solution was added to the second solution using mixing with an Ultra Turrax T25 (IKA) at 6000 rpm for 5 minutes while maintaining the temperature of the emulsion between 20 and 30° C. 10 ml water was added followed by evaporation of the mixture under reduced pressure to 25 g. The polymerization was allowed to continue at room temperature for 24 hours.

### γ-Benzyl Glutamate as (Co)Monomer in the Shell (INVCAP-12 and INVCAP-13)

### The Synthesis of INVCAP-12

1.5 g L-γ-benzyl glutamate N-carboxy anhydride, 0.75 g L-leucine N-carboxy anhydride and 0.75 g D-leucine N-carboxy anhydride were dissolved in 18 ml ethyl acetate. The solution was filtered over a 1.7 micron filter. 0.294 g Crosslinker-1, 0.1 g Tracer-1 and 2.26 g glyceryl tricaprate were added.

A second solution was prepared by dissolving 0.68 g Mowiol 4 88, 0.255 g Marlon A365 and 0.111 g tris(2-aminoethyl) amine in 30 ml water.

The first solution was added to the second solution using mixing with an Ultra Turrax T25 (IKA) at 6000 rpm for 5 minutes while maintaining the temperature of the emulsion between 20 and 30° C. 10 ml water was added followed by evaporation of the mixture under reduced pressure to 25 g. The polymerization was allowed to continue at room temperature for 24 hours.

### The Synthesis of INVCAP-13

0.6 g L-γ-benzyl glutamate N-carboxy anhydride, 0.6 g L-leucine N-carboxy anhydride, 0.6 g D-leucine N-carboxy anhydride, 0.6 g L-phenylalanine N-carboxy anhydride and 0.6 g D-phenylalanine N-carboxy anhydride were dissolved in 18 ml ethyl acetate. The solution was filtered over a 1.7 micron filter. 0.313 g Crosslinker-1, 0.1 g Tracer-1 and 2.32 g glyceryl tricaprate were added.

A second solution was prepared by dissolving 0.68 g Mowiol 4 88, 0.255 g Marlon A365 and 0.118 g tris(2-aminoethyl) amine in 30 ml water.

The first solution was added to the second solution using mixing with an Ultra Turrax T25 (IKA) at 6000 rpm for 5 minutes while maintaining the temperature of the emulsion between 20 and 30° C. 10 ml water was added followed by evaporation of the mixture under reduced pressure to 25 g. The polymerization was allowed to continue at room temperature for 24 hours.

### Characterization of INVCAP-4 to INVCAP-13

The inventive capsules INVCAP-4 to INVCAP-13 were characterized by fluorescence imaging and centrifugation as disclosed in Example 1. Based on this analysis, it was proven that glyceryl tricaprate was fully encapsulated in all cases.

### C.5. Example 3

This examples illustrates the use of a different crosslinker in the synthesis of the capsules according to the present invention. Trifunctional isocyanates were selected as crosslinker in the synthesis of INVCAP-14 and INVCAP-15.

### The Synthesis of INVCAP-14

0.75 g L-leucine N-carboxy anhydride, 0.75 g D-leucine N-carboxy anhydride, 0.75 g L-phenylalanine N-carboxy anhydride and 0.75 g D-phenylalanine N-carboxy anhydride were dissolved in 18 ml ethyl acetate. The solution was filtered over a 1.7 micron filter. 0.831 Takenate D120N, 0.1 g Tracer-1 and 2.59 g glyceryl tricaprate were added.

A second solution was prepared by dissolving 0.692 g Mowiol 4 88, 0.259 g Marlon A365 and 0.127 g tris(2-aminoethyl) amine in 30 ml water.

The first solution was added to the second solution using mixing with an Ultra Turrax T25 (IKA) at 6000 rpm for 5 minutes while maintaining the temperature of the emulsion between 20 and 30° C. 10 ml water was added followed by evaporation of the mixture under reduced pressure to 30 g. The polymerization was allowed to continue at room temperature for 24 hours.

### The Synthesis of INVCAP-15

0.75 g L-leucine N-carboxy anhydride, 0.75 g D-leucine N-carboxy anhydride, 0.75 g L-phenylalanine N-carboxy anhydride and 0.75 g D-phenylalanine N-carboxy anhydride were dissolved in 18 ml ethyl acetate. The solution was filtered over a 1.7 micron filter. 0.555 Desmodur N75BA, 0.1 g Tracer-1 and 2.59 g glyceryl tricaprate were added.

A second solution was prepared by dissolving 0.692 g Mowiol 4 88, 0.259 g Marlon A365 and 0.127 g tris(2-aminoethyl) amine in 30 ml water.

The first solution was added to the second solution using mixing with an Ultra Turrax T25 (IKA) at 6000 rpm for 5 minutes while maintaining the temperature of the emulsion between 20 and 30° C. 10 ml water was added followed by evaporation of the mixture under reduced pressure to 30 g. The polymerization was allowed to continue at room temperature for 24 hours.

### Characterization of INVCAP-14 and INVCAP-15

The inventive capsules INVCAP-14 to INVCAP-15 were characterized by fluorescence imaging and centrifugation as disclosed in Example 1. Based on this analysis, it was proven that glyceryl tricaprate was fully encapsulated in all cases.

### C.6. Example 4:

This example illustrates the applicability of various colloid stabilizing mechanism in the synthesis of the capsules according to the present invention by replacing the non-ionic polymeric stabilizer and anionic surfactant used in the previous examples by a cationic co-reactive surfactant in the capsule synthesis as illustrated by the synthesis of a cationic self-dispersing capsule.

### The Synthesis of INVCAP-16

0.75 g L-leucine N-carboxy anhydride, 0.75 g D-leucine N-carboxy anhydride, 0.75 g L-phenylalanine N-carboxy anhydride and 0.75 g D-phenylalanine N-carboxy anhydride were dissolved in 25 ml ethyl acetate. 0.336 g Crosslinker-1, 0.1 g Tracer-1 and 2.50 g glyceryl tricaprate were added.

A second solution was prepared by dissolving 1.01 g of CATSURF-1 in 30 ml water.

The first solution was added to the second solution using mixing with an Ultra Turrax T25 (IKA) at 6000 rpm for 5 minutes while maintaining the temperature of the emulsion between 20 and 30° C. 10 ml water was added followed by evaporation of the mixture under reduced pressure to 30 g. The polymerization was allowed to continue at room temperature for 24 hours.

### Characterization of INVCAP-16

The inventive capsules INVCAP-16 was characterized by fluorescence imaging and centrifugation as disclosed in Example 1. Based on this analysis, it was proven that glyceryl tricaprate was fully encapsulated in INVCAP-16.

### C.7. Example 5

This example illustrates the option to isolate the capsules according to the present invention using freeze drying.

A first solution was made by dissolving 1.5 g D,L-phenylalanine N-carboxy anhydride, 0.75 g L-leucine N-carboxy anhydride and 0.75 g D-Leucine N-carboxy anhydride in 18 ml ethyl acetate. 0.336 g Crosslinker-1, 2.309 g glyceryl tricaprate and 0.1 g were added.

A second solution was prepared by dissolving 0.692 g Mowiol 4 88, 0.259 g Marlon A365 and 0.127 g tris(2-aminoethyl) amine in 30 ml water.

The first solution was added to the second solution using mixing with an Ultra Turrax (IKA) at 6000 rpm for 5 minutes, while maintaining the temperature of the emulsion between 20 and 30° C. The ethyl acetate was removed under reduced pressure and the weight of the dispersion was adjusted to 30 g. The polymerization was allowed to continue at room temperature for 24 hours.

The capsule was isolated by freeze drying. The re-dispersibility of the capsules was evaluated by re-dispersing a sample of the isolated capsules in water using sonification, using a Sona Vibra Cell at an output of 19-21 watts, an amplitude of 100 for 5 seconds. The degree of dispersion was evaluated using a light microscope at a magnification of 63× and the image was compared with the microscopical analysis of the original dispersion, obtained after synthesis. Both images showed the same degree of dispersion. After re-dispersion no additional oversizers or clusters could be detected.

1.-15. (canceled)

16. A capsule consisting of a polymeric shell surrounding a core, the core comprises an organic compound, the polymeric shell comprises a poly(amino acid) and is obtainable by interfacial polymerization of a N-carboxy-anhydride monomer according to general structure I: 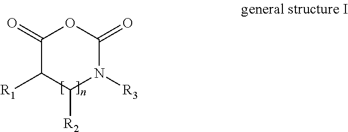
wherein
n represents 0 or 1
R₁, R₂ and R₃ are selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group R₁, R₂ and R₃ may represent the necessary atoms to form a five to eight membered ring.

17. The capsule according to claim 16, wherein the organic compound has an octanol-water partition coefficient, expressed as log K_{ow} of 0.3 or more.

18. The capsule according to claim 16, wherein the organic compound is selected from the group consisting of marine oils, vegetable oils, essential oils, fragrances, flavours, insect repellents, flame retardants, active pharmaceutical ingredients and agrochemicals.

19. The capsule according to claim 17, wherein the organic compound is selected from the group consisting of marine oils, vegetable oils, essential oils, fragrances, flavours, insect repellents, flame retardants, active pharmaceutical ingredients and agrochemicals.

20. The capsule according to claim 16, further having an average particle size from 0.07 μm to 5 μm.

21. The capsule according to claim 16, wherein the polymeric shell comprises a crosslinker.

22. The capsule according to claim 17, wherein the polymeric shell comprises a crosslinker.

23. The capsule according to claim 16, wherein the polymeric shell comprises a dispersing group selected from the group consisting of a carboxylic acid or a salt thereof, a sulfonic acid or salt thereof, a phosphoric acid ester or a salt thereof, a phosphonic acid or salt thereof a protonated amine, a protonated nitrogen containing heteroaromatic compound, a quaternized tertiary amine, a N-quaternized heteroaromatic group, a sulfonium and a phosphonium.

24. The capsule according to claim 17, wherein the polymeric shell comprises a dispersing group selected from the group consisting of a carboxylic acid or a salt thereof, a sulfonic acid or salt thereof, a phosphoric acid ester or a salt thereof, a phosphonic acid or salt thereof a protonated amine, a protonated nitrogen containing heteroaromatic compound, a quaternized tertiary amine, a N-quaternized heteroaromatic group, a sulfonium and a phosphonium.

25. The capsule according to claim 16, wherein the poly(amino acid) comprises an L-amino acid and a D-amino acid.

26. The capsule according to claim 17, wherein the poly(amino acid) comprises an L-amino acid and a D-amino acid.

27. The capsule according to claim 18, wherein the organic compound is selected from the group consisting of an anti-cancer drug, a vaccine, a peptide, a protein and a sonosensitizer.

28. A pharmaceutical composition comprising the capsule as claimed in claim 27 and a pharmaceutical carrier or excipient.

29. A consumer product comprising the capsule as defined in claim 16, wherein the consumer product is selected from the group consisting of a shampoo, a hair conditioner, a hair rinse, a hair refresher, a hair fixative or styling aid, a hair bleach, a hair dye or colorant, a soap, a body wash, a cosmetic preparation, an all-purpose cleaner, a bathroom cleaner, a floor cleaner, a window cleaner, a bath tissue, a paper towel, a disposable wipe, a diaper rash cream or balm, a baby powder, a diaper, a bib, a baby wipe, an oral care product, a tooth paste, an oral rinse, an tooth whitener, a denture adhesive, a chewing gum, a breath freshener, an orally dissolvable strips, a chewable candy, a hard candy, a hand sanitizer, an anti-inflammatory balm, an anti-inflammatory ointment, an anti-inflammatory spray, a health care device, a dental floss, a toothbrush, a tampon, a feminine napkin, a personal care product, a sunscreen lotion, a sunscreen spray, a wax-based deodorant, a glycol type deodorant, a soap type deodorant, a facial lotion, a body lotion, a hand lotion, a body powder, a shave cream, a bath soak, an exfoliating scrub, a foot cream, a facial tissue, a cleansing wipe, a fabric care product, a fabric softener, a fabric refresher, an ironing water, a liquid laundry detergent, a liquid dish detergent, an automatic dish detergent, a unit dose tablet or capsule, a scent booster, a drier sheet, a fine fragrance, a solid perfume, a powder foundation, a liquid foundation, an eye shadow, a lipstick or lip balm, an Eau De Toilette product, a deodorant, a rug deodorizer, a candle, a room deodorizer, a disinfectant, an anti-perspirant, an roll-on product, and an aerosol product.

30. A method of preparing the capsules as defined in claim 16, comprising the steps of:
a) dissolving a N-carboxy-anhydride monomer according to general structure I and an organic compound in a water immiscible solvent; and
b) dissolving a polymerization initiator in an aqueous liquid; and
c) emulsifying the solution obtained in step a) into the aqueous liquid; and
d) optionally evaporating the water immiscible solvent; and
e) polymerizing the N-carboxy-anhydride monomer according to general structure I.

31. The method of preparing the capsules according to claim 30, wherein the organic compound has an octanol-water partition coefficient, expressed as log K_{ow} of 0.3 or more.

32. The method of preparing the capsules according to claim 30, wherein a surfactant or hydrophilic polymer is added to the aqueous liquid.

33. The method of preparing the capsules according to claim 30, wherein a crosslinker is added to the water immiscible solvent in step a).

34. The method of preparing the capsules according to claim 30, wherein the polymerization initiator is a di- or multifunctional primary or secondary amine.
